(19) United States
(12) Patent Application Publication
(10) Pub. No.: US 2013/0312756 A1
(43) Pub. Date: Nov. 28, 2013

(54) AIRTIGHT SUCTION DEVICE AND AIR SUPPLY ASSEMBLY THEREOF

(71) Applicant: Hsuan-Chiao HO, Tongluo Township (TW)

(72) Inventor: Hsuan-Chiao HO, Tongluo Township (TW)

(73) Assignee: Pacific Hospital Supply Co., Ltd.

(21) Appl. No.: 13/480,325

(22) Filed: May 24, 2012

(51) Int. Cl. A61M 1/00; A61M 16/00

(52) U.S. Cl. 128/205.12

(57) ABSTRACT

An air supply assembly of an airtight suction device includes: a tubular body, an oxygen supply manifold in communication with the tubular body; a scraper fixed in the tubular body; and an air supply unit provided in the tubular body and located between the oxygen supply manifold and the scraper. An airtight suction device can further includes a tubular body, an oxygen supply manifold, a first scraper, a second scraper, and an air supply unit.

### BACKGROUND OF THE INVENTION

1. Field of the Invention

The present invention relates to an airtight suction device and an air supply assembly thereof. In particular, the present invention relates to an airtight suction device having an air supply unit and an air supply assembly thereof.

2. Description of Prior Art

A suction device is used in a tracheostomy patient. The tracheostomy is a medical operation for providing a cut-out on the neck of a patient to thereby allow a tracheostomy tube to be inserted therein, whereby external air can enter the lungs of the patient via the tracheostomy tube. Since the external air directly enters the lungs via the tracheostomy tube without passing through the nasal cavity of the patient, the external air is not wetted by the warm air in the nasal cavity. As a result, the dry external air will cause the increase of secretions and sputum in the trachea. Thus, a suction tube 30 needs to be inserted into the respiratory tract for sucking the secretions and the sputum frequently.

Please refer to FIG. 1. The existing suction device is connected to an oxygen conduit 20 and a connecting tube 10. The oxygen conduit 20 is connected to an oxygen supply source, so that the oxygen supply source can supply oxygen through the oxygen conduit 20 and the connecting pipe 10 into the patient. The suction tube 30 is inserted into the connecting tube 10 to suck the waste liquid of the patient including secretions and sputum in the respiratory tract. When the suction tube 30 is to be replaced, the end of the suction tube 30 inserted into the patient (referred to as the “patient end” hereinafter) is drawn out of the patient and inserted into a cleaning chamber 60. At this time, cleaning water coming from a cleaning tube 40 is filled into the cleaning chamber 60, so that the waste liquid can be exhausted from the suction tube 30 by means of the negative pressure generated by the suction tube 30.

The conventional suction device has the following problem. Since the cleaning chamber 60 is not completely separated from the oxygen conduit 20, a portion of oxygen will be drawn out of the suction tube 30 when the waste liquid is drawn.

### SUMMARY OF THE INVENTION

The present invention is to provide an air supply assembly of an airtight suction device, in which a tubular body of the airtight suction device has an air supply unit. When the waste liquid is exhausted to the outside by means of a negative pressure generated by a suction tube, the air outside the tubular body will enter the tubular body via the air supply unit, thereby reducing the amount of oxygen that is exhausted to the outside.

The present invention provides an air supply assembly of an airtight suction device, including: a tubular body; an oxygen supply manifold in communication with the tubular body; a scraper fixed into the tubular body, a distance being formed between the scraper and the oxygen supply manifold along the axial direction of the tubular body; and an air supply unit provided in the tubular body and located between the oxygen supply manifold and the scraper.

Preferably, in the air supply assembly of the airtight suction device, the air supply unit is a through-hole provided in the tubular body.

Preferably, in the air supply assembly of the airtight suction device, the air supply unit is a conical through-hole provided in the tubular body. The diameter of the air supply unit outside the tubular body is larger than the diameter of the air supply unit inside the tubular body.

Preferably, in the air supply assembly of the airtight suction device, the air supply unit is a check valve provided in the tubular body for allowing air to flow from the outside of the tubular body to the inside of the tubular body.

Preferably, the air supply assembly of the airtight suction device further includes another scraper fixed into the tubular body and located between the oxygen supply manifold and the scraper. The air supply unit is provided between these two scrapers.

The present invention is to provide an airtight suction device, in which an oxygen supply manifold is connected to an oxygen conduct and a tubular body is provided to have an air supply unit. When the waste liquid is exhausted to the outside by means of a negative pressure generated by a suction tube, the air outside the tubular body will flow into the tubular body via the air supply unit, thereby reducing the amount of oxygen that is exhausted to the outside.

The present invention provides an airtight suction device, which is put on a suction tube and includes a tubular body, an oxygen supply manifold, a first scraper, a second scraper, and an air supply unit.

The tubular body comprises a connecting section and an exhaust section opposite to the connecting section. The oxygen supply manifold is in communication with the tubular body and located between the connecting section and the exhaust section. The first scraper is fixed into the exhaust section and provided with a first through-hole. The second scraper is fixed into the exhaust section and located between the oxygen supply manifold and the first scraper. The second scraper is provided with a second through-hole. The suction tube extends from the exhaust section to pass through the second through-hole and the first through-hole. The air supply unit is provided in the tubular body and located between the oxygen supply manifold and the first scraper.

Preferably, the airtight suction device further comprises a cleaning manifold in communication with the exhaust section and located between the first scraper and the second scraper.

Preferably, in the airtight suction device, the air supply unit is a through-hole provided in the tubular body.

Preferably, in the airtight suction device, the air supply unit is a conical through-hole provided in the tubular body. The diameter of the air supply unit outside the tubular body is larger than the diameter inside the tubular body.

Preferably, in the airtight suction device, the air supply unit is a check valve provided in the tubular body for allowing air to flow from the outside of the tubular body to the inside of the tubular body.

Preferably, in the airtight suction device, the air supply unit is located between the first scraper and the second scraper.

In comparison with prior art, the present invention has the following advantageous features. According to the airtight suction device of the present invention, when the suction tube generates a negative pressure to suck the waste liquid, the air outside the airtight suction device enters the space between the first scraper and the second scraper via the air supply unit. Thus, the amount of oxygen exhausted to the outside via the first through-hole can be reduced greatly.

### BRIEF DESCRIPTION OF DRAWING

FIG. 1 is a cross-sectional view of a conventional suction device;

FIG. 2 is a cross-sectional view showing the airtight suction device according to a first embodiment of the present invention;

FIG. 3 is a cross-sectional view showing another aspect of the airtight suction device according to the first embodiment of the present invention;

FIG. 4 is a cross-sectional view showing the operating state of the airtight suction device according to the first embodiment of the present invention;

FIG. 5 is a cross-sectional view showing a cleaning chamber of an air supply assembly in the airtight suction device according to a second embodiment of the present invention; and

FIG. 6 is a cross-sectional view showing a cleaning chamber of an air supply assembly in the airtight suction device according to a third embodiment of the present invention.

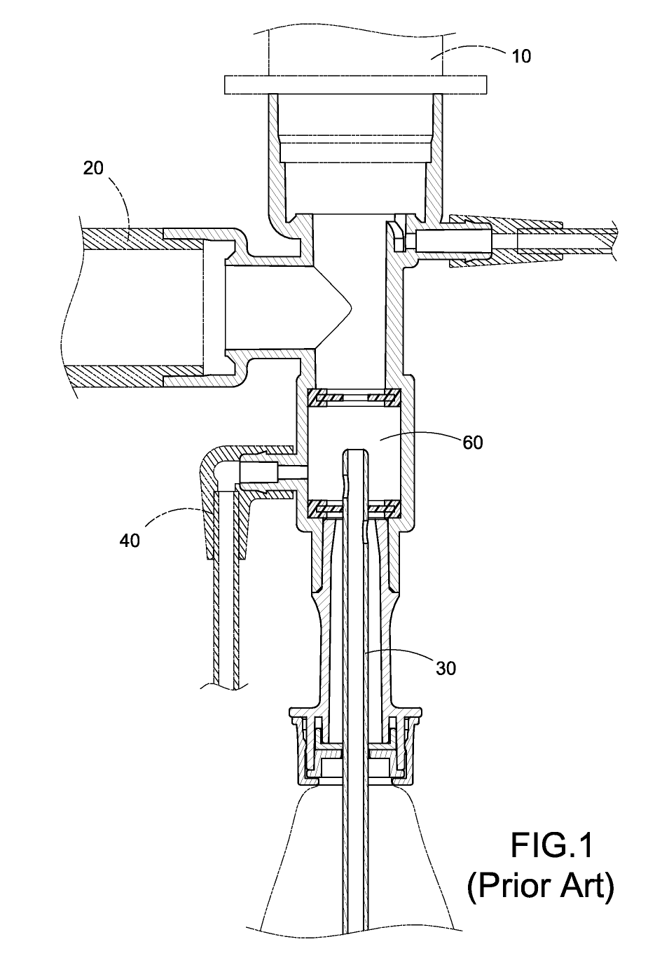

### DETAILED DESCRIPTION OF THE INVENTION

The detailed description and technical contents of the present invention will become apparent with the following detailed description accompanied with related drawings. It is noteworthy to point out that the drawings is provided for the illustration purpose only, but not intended for limiting the scope of the present invention.

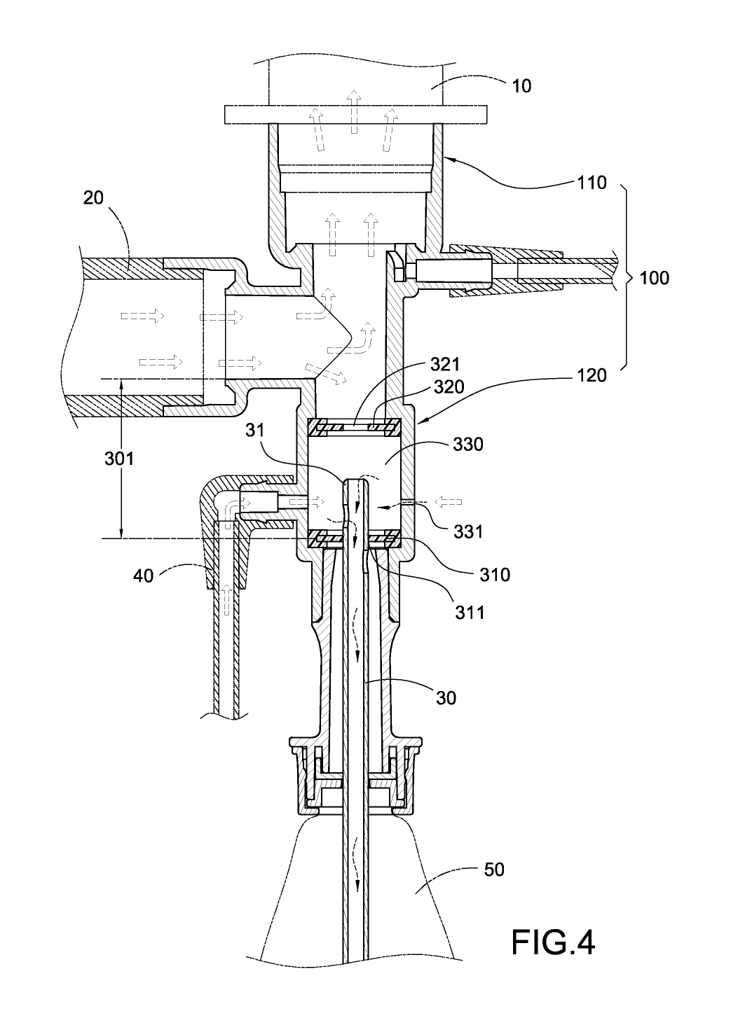

The first embodiment of the present invention provides an airtight suction device, which is put on a suction tube 30 shown in FIG. 4. The suction tube 30 is an elongate tube in which a negative pressure is generated. The suction tube 30 has a patient end 31 which is an open end inserted into a patient, so that the waste liquid of the patient can be drawn out via the negative pressure.

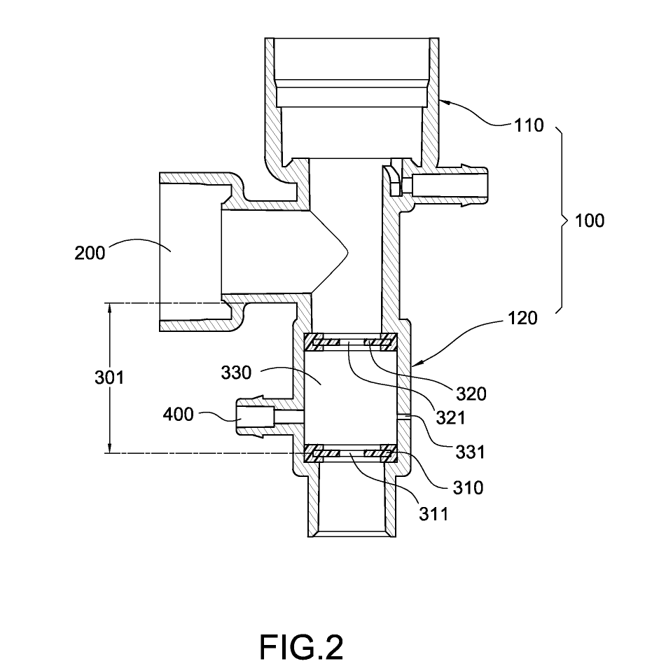

Please refer to FIG. 2. The airtight suction device of the present invention includes a tubular body 100, an oxygen supply manifold 200, a first scraper 310, a second scraper 320, an air supply unit 331, and a cleaning manifold 400.

The tubular body 100 has two open ends. These two opposite open ends act as a connecting section 110 and an exhaust section 120 respectively. The connecting section 110 is connected to a connecting tube 10 (FIG. 4). The connecting tube 10 is connected to a portion on which a cut-out is generated in communication with the bronchia of the patient. The exhaust section 120 is connected to a collecting bag 50 (FIG. 4).

One end of the oxygen supply manifold 200 is in communication with a branch of the tubular body 100 and located between the connecting section 110 and the exhaust section 120. The other end of the oxygen supply tube 200 is connected to an oxygen conduit 20 as shown in FIG. 4. Oxygen is inputted from the oxygen conduit 20 into the tubular body 100 and into the patient via the connecting tube 10.

The first scraper 130 is located in the exhaust section 120 to abut against the inner wall of the exhaust section 120 and thus fixed in the exhaust section 120. A distance 301 is formed between the first scraper 310 and the oxygen supply manifold 200 in the axial direction of the exhaust section 120. The first scraper 310 is provided with a first through-hole 311. The diameter of the first through-hole 311 is substantially the same as the outer diameter of the suction tube 30, so that the suction tube 30 can exactly pass through the first through-hole 311. Preferably, the present invention may be provided with single scraper (having the same function and structure as those of the first scraper 310) without another scraper (like the second scraper 320). A distance 301 is formed between the scraper (the first scraper 310) and the oxygen supply manifold 200 in the axial direction of the exhaust section 120.

The second scraper 320 is located in the exhaust section 120 to abut against the inner wall of the exhaust section 120 and thus fixed in the exhaust section 120. The first scraper 310 and the second scraper 320 are in parallel to each other. The second scraper 320 is provided between the oxygen supply manifold 200 and the first scraper 310, so that the first scraper 310 and the second scraper 320 are separated from each other in the exhaust section 120 to thereby form a cleaning chamber 330. The second scraper 320 is provided with a second through-hole 321. The diameter of the second through-hole 321 is substantially the same as that of the suction tube 30, so that the suction tube 30 can exactly pass through the second through-hole 321. The suction tube 30 penetrates from the collecting bag 50 into the exhaust section 120 to sequentially pass through the first through-hole 311, the second through-hole 321 and the connecting section 110 until the patient end 31 is inserted into the connecting tube 10. In this way, the waste liquid of the patient can be drawn out to flow into the collecting bag 50.

One end of the cleaning manifold 400 is in communication with the exhaust section 120 and located between the oxygen supply manifold 200 and the first scraper 310. In the present embodiment, the cleaning manifold 400 is in communication with the exhaust section 120 and located between the first scraper 310 and the second scraper 320. The other end of the cleaning manifold 400 is connected to a water tube 40 for filling water in the cleaning chamber 330.

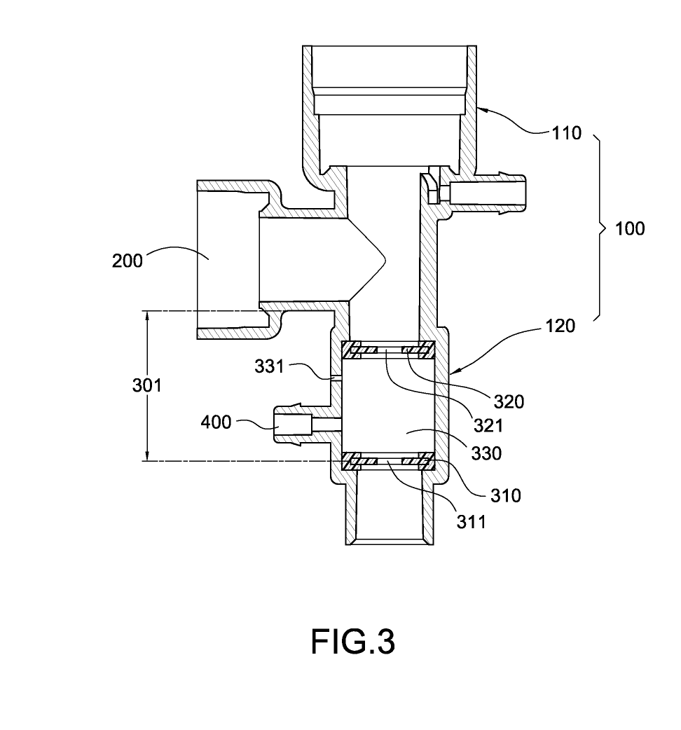

Please refer to FIGS. 2 and 3. In the present embodiment, the air supply unit 331 is preferably configured as a through-hole, which is provided on the wall of the exhaust section 120 of the tubular body 100 and located between the first scraper 310 and the second scraper 320 for supplementing air in the cleaning chamber 330. Preferably, the air supply unit 331 is provided on the wall of the exhaust section 120 and located between the oxygen supply manifold 200 and the first scraper 310. For example, in FIG. 2, the air supply unit 331 and the cleaning manifold 400 are provided in the cleaning chamber 330 in such a manner that they are opposite to each other. For example, in FIG. 3, the air supply unit 331 and the cleaning manifold 400 are provided in the cleaning chamber 330 in such a manner that they are arranged close to each other,

Please refer to FIG. 4. When the suction tube 30 is drawn out of the connecting tube 10, the patient end 31 is drawn into the cleaning chamber 330. When the patient end 31 passes through the first scraper 310 and the second scraper 320, the waste liquid adhered on the outer surface of the patient end 31 will be scraped away by the first scraper 310 and the second scraper 320 to stay in the first through-hole 311 and the second through-hole 321. Water is filled from the cleaning manifold 400 of the water tube 40 into the cleaning chamber 330 to thereby clean the patient end 31, the first scraper 310 and the second scraper 320. The used cleaning water is drawn from the suction tube 30 into the collecting bag 50. The suction tube 30 provides a negative pressure to the cleaning chamber 330 to thereby draw the waste liquid out of the cleaning chamber 330. At this time, the air outside the cleaning chamber 330 enters the cleaning chamber 330 between the first scraper 310 and the second scraper 320 via the air supply unit 331. Therefore, the pressure difference on both sides of the second scraper 320 (between the connecting section 110 and the cleaning chamber 330) is decreased, so that the amount of oxygen passing through the second through-hole 321 and flowing enter the cleaning chamber 330 can be reduced.

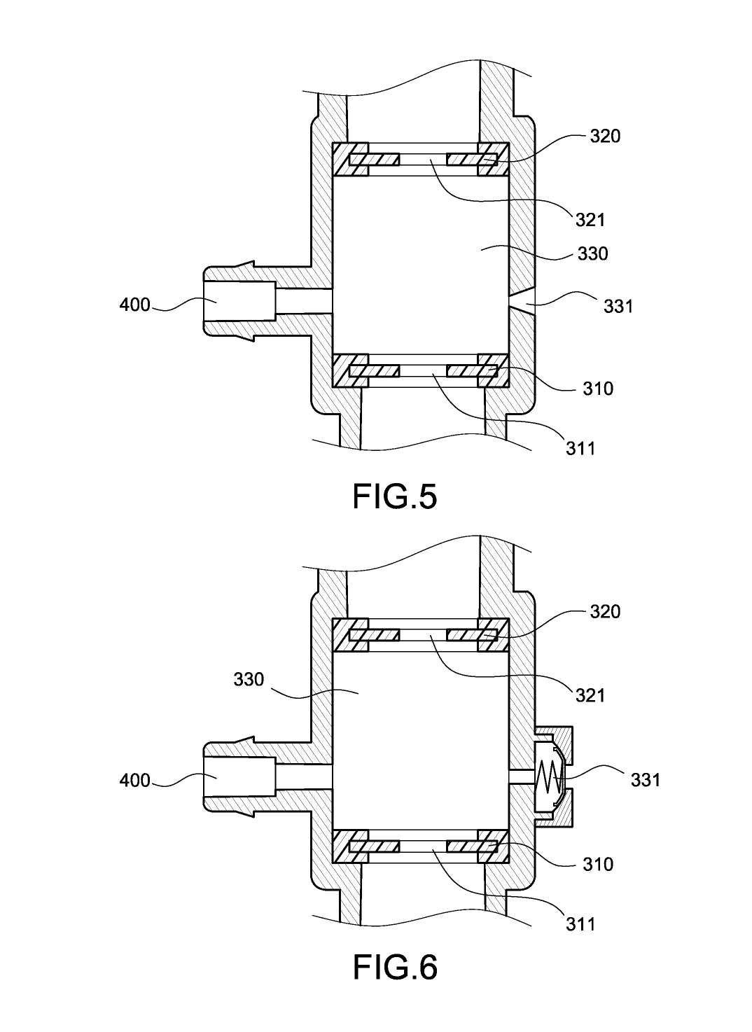

Please refer to FIG. 5. The second embodiment of the present invention provides an air supply assembly of an airtight suction device, which is used to put on a suction tube 30. The suction tube 30 has a structure as mentioned in the first embodiment.

The air supply assembly of the airtight suction device according to the present invention includes a tubular body 100, an oxygen supply manifold 200, a first scraper 310, a second scraper 320, a cleaning manifold 400, and an air supply unit 331. In the present embodiment, the structures and the relationships of connection of the tubular body 100, the oxygen supply manifold 200, the first scraper 310, the second scraper 320, and the cleaning manifold 400 are the substantially the same as those of the first embodiment, so that the redundant description is omitted for simplicity. The difference between the second embodiment and the first embodiment lies in that: the air supply unit 331 is provided on the wall of the exhaust section 120 of the tubular body 100 and located between the first scraper 310 and the second scraper 320 for supplying air in the cleaning chamber 330. Preferably, the air supply unit 331 is a conical through-hole. The diameter of the air supply unit 331 on the outer surface of the exhaust section 120 is larger than the diameter of the air supply unit 331 on the inner surface of the exhaust section 120, so that the air can only flow from the outside of the exhaust section 120 into the cleaning chamber 330 without flowing out of the cleaning chamber 330.

Please refer to FIG. 6. The third embodiment of the present invention provides an air supply assembly of an airtight suction device, which is used to put on a suction tube 30. The suction tube 30 has a structure substantially the same as the first embodiment.

The air supply assembly of the airtight suction device according to the present invention includes a tubular body 100, an oxygen supply manifold 200, a first scraper 310, a second scraper 320, a cleaning manifold 400, and an air supply unit 331. In the present embodiment, the structures and the relationships of connection of the tubular body 100, the oxygen supply manifold 200, the first scraper 310, the second scraper 320, and the cleaning manifold 400 are the substantially the same as those of the first embodiment, so that the redundant description is omitted for simplicity. The difference between the third embodiment and the first embodiment lies in that: the air supply unit 331 is provided on the wall of the exhaust section 120 of the tubular body 100 and located between the first scraper 310 and the second scraper 320 for supplying air in the cleaning chamber 330. Preferably, the air supply unit 331 is an one-way check valve, which allows air to flow from the outside of the exhaust section 120 to the inside of the exhaust section 120. By this arrangement, the air can only flow from the outside of the exhaust section 120 into the cleaning chamber 330 without flowing out of the cleaning chamber 330.

According to the airtight suction device and the air supply assembly of the present invention, the wall of the cleaning chamber 330 is provided with the air supply unit 331. Thus, when the suction tube 30 provides a negative pressure to draw out the waste liquid, the air outside the airtight suction device can pass through the air supply unit 331 to enter the cleaning chamber 330, thereby reducing the amount of oxygen exhausted from the first through-hole 311. Therefore, the problem of the conventional suction device that a great amount of oxygen will be exhausted when the waste liquid is drawn out can be solved.

Although the present invention has been described with reference to the foregoing preferred embodiments, it will be understood that the invention is not limited to the details thereof. Various equivalent variations and modifications can still occur to those skilled in this art in view of the teachings of the present invention. Thus, all such variations and equivalent modifications are also embraced within the scope of the invention as defined in the appended claims.

What is claimed is:

1. An air supply assembly of an airtight suction device, including:
a tubular body;
an oxygen supply manifold in communication with the tubular body;
a scraper fixed into the tubular body, a distance being formed between the scraper and the oxygen supply manifold in the axial direction of the tubular body; and
an air supply unit provided in the tubular body and located between the oxygen supply manifold and the scraper.

2. The air supply assembly of an airtight suction device according to claim 1, wherein the air supply unit is a through-hole provided in the tubular body.

3. The air supply assembly of an airtight suction device according to claim 1, wherein the air supply unit is a conical through-hole provided in the tubular body, the diameter of the air supply unit on the outer surface of the exhaust section is larger than the diameter of the air supply unit on the inner surface of the exhaust section.

4. The air supply assembly of an airtight suction device according to claim 1, wherein the air supply unit is a check valve provided in the tubular body for allowing air to flow from the outside of the tubular body to the inside of the tubular body.

5. The air supply assembly of an airtight suction device according to claim 1, further including another scraper fixed in the tubular body and located between the oxygen supply manifold and the scraper, the air supply unit being provided between the scraper and the another scraper.

6. An airtight suction device, configured to be put on a suction tube and including:
a tubular body comprising a connecting section and an exhaust section opposite to the connecting section;
an oxygen supply manifold in communication with the tubular body and located between the connecting section and the exhaust section;
a first scraper fixed in the exhaust section, the first scraper being provided with a first through-hole;
a second scraper fixed in the exhaust section and located between the oxygen supply manifold and the first scraper, the second scraper being provided with a second through-hole, the suction tube penetrating the exhaust section to pass through the second through-hole and the first through-hole; and
an air supply unit provided in the tubular body and located between the oxygen supply manifold and the first scraper.

7. The airtight suction device according to claim 6, further including a cleaning manifold in communication with the exhaust section and located between the first scraper and the second scraper.

8. The airtight suction device according to claim 6, wherein the air supply unit is a through-hole provided in the tubular body.

9. The airtight suction device according to claim 6, wherein the air supply unit is a conical through-hole provided in the tubular body, the diameter of the air supply unit on the outer surface of the exhaust section is larger than the diameter of the air supply unit on the inner surface of the exhaust section.

10. The airtight suction device according to claim 6, wherein the air supply unit is a check valve provided in the tubular body for allowing air to flow from the outside of the tubular body to the inside of the tubular body.

11. The airtight suction device according to claim 6, wherein the air supply device is located between the first scraper and the second scraper.
